(19) United States
(12) Patent Application Publication
(10) Pub. No.: US 2014/0114132 A1
(43) Pub. Date: Apr. 24, 2014

(54) DEVICE SUPPORTING TRACHEAL TUBE

(71) Applicant: Andrea De Domenico, Rome (IT)

(72) Inventor: Andrea De Domenico, Rome (IT)

(21) Appl. No.: 14/126,753

(22) PCT Filed: Jun. 12, 2012
(86) PCT No.: PCT/EP2012/061135

§ 371 Date: Dec. 16, 2013

(30) Foreign Application Priority Data
Jun. 14, 2011 (EP) 11425158.0

(51) Int. Cl. A61M 16/04; A61B 1/04; A61B 1/00; A61B 1/267

(52) U.S. Cl. CPC A61M 16/0488; A61B 1/267; A61B 1/04; A61B 1/00158
USPC 600/188; 600/194; 128/200.26

(57) ABSTRACT

A device for supporting a tracheal tube during tracheal intubation. The device is applied to the blade of a laryngoscope, or similar instrument. It includes one or more planar elements, each element having a profile adapted to the curvature of the blade a the location in which it is placed. On command, each element protrudes out from the blade through a longitudinal groove, in order to create one or more fixed transversal walls. Once the walls are created, the tracheal tube is then inserted into the laryngoscope and its far end is moved across the walls, and towards the trachea of the patient. The device also includes a set of mechanical gears that are controlled by a sliding button such that moving the button from a back position to a front position moves each of the planar elements from the position within the blade to the protruding position.

### CROSS-REFERENCE TO RELATED APPLICATIONS

This patent application is a national phase entry under 35 U.S.C. §371 of PCT International Patent Application PCT/EP2012/0061135, filed Jun. 12, 2012, designating the United States and published in English as International Patent Publication WO 2012/171926 A1 on Dec. 20, 2012, which claims the benefit, under Article 8 of the PCT, of European Patent Application Serial No. 11425158.0, filed Jun. 14, 2011, the benefit and entire disclosure of each of which is hereby incorporated herein by this reference.

### FIELD OF THE INVENTION

The present invention concerns a medical device, preferably applied to the blade of a laryngoscope, or an Airtraq® videolaryngoscope, or a LMA® videolaryngoscope, or other similar instrument, and particularly suitable as a support to the procedure of tracheal intubation of a patient. In particular, the device includes one or more planar elements, each having a profile that is perfectly adapted to the curvature of said blade in which it is placed, and from which it can protrude out, on command, through a groove, in order to achieve one or more respective fixed transversal walls. A tracheal tube positioned with its far end across said one or more transversal walls, can then be curved and addressed towards the patient's trachea.

### BACKGROUND

Direct laryngoscopy is a technique that permits one to visualize the laryngeal structures of a patient in a clear and straight way. It is achieved using an instrument called a laryngoscope. This technique has made it possible, for anesthetists operating around the world, to define a standard procedure of tracheal intubation in order to place a tube in a trachea, by which a patient can receive oxygen and at the same time can have protected air ways during the anesthesia.

Despite the improvement of such technique, obtained at a global level by years of clinical practice, and despite the different shapes of laryngoscopic blades that have been designed and are available on the market, in some cases, direct laryngoscopy remains impossible, either because of particular anatomical parts in a given person or because of particular situations that can happen in an emergency.

For example, direct laryngoscopy is not possible when the alignment of the patient's three axes (oral axis, pharyngeal axis and laryngeal axis) cannot be achieved by the standard actions that a physician knows and can perform. Therefore, for a long time there was a widely recognized need to find some alternative instruments and techniques that would permit an optimal visualization of a glottis, even in cases where direct laryngoscopy results are not effective or are impossible.

When new technologies become available, with new advanced materials, the laryngeal structures had more chances to be properly visualized, even in indirect ways, by the so called indirect laryngoscopy.

Therefore, the techniques of indirect laryngoscopy made it possible to visualize the laryngeal structures clearly, using a camera, or an optical fiber, or through a system of prisms with distal elements that can be placed close to the glottis.

Nowadays, a large set of instruments are available, permitting the handling of even hard situations, and many of these instruments embed indirect laryngoscopy based systems.

With reference to devices that, at the present time, permit the achievement of the indirect laryngoscopy technique, except the flexible and stiff fiberscope, or Bonfils fiberscope, they belong to two possible classes:
the videolaryngoscopes (Glidescope, McGrath, and C-MAC);
the tunneled systems (Upscher Scope, Bullard laryngoscope, Airtraq, Pentax AWS, and A.P. Advance LMA).

Besides the different visual, optical or video system, each device belongs to the first or the second class, according to the procedure of use of the same device, depending on when the operator can see the laryngeal structures and can insert the endotracheal tube up to its proper position in trachea.

Devices in the first class, Glidescope, McGrath, and C-MAC, once the indirect laryngoscopy is possible, because of the blade's shape, usually require that the operator properly shape the endotracheal tube, before inserting it. Therefore, the tube's profile is adapted, according to a specific angle, by inserting a stiff stylet inside, so that the final shape allows the routing of the tube beyond the tongue, towards the patient's trachea.

Instead, devices in the second class, like i.e. Airtraq, Pentax AWS, and A.P. Advance LMA, contain the endotracheal tube in a tunnel that is integrated into the blade, so that guidance can be achieved all along the path, until the far end of the tube exits the tunnel directly on the proper position in trachea. Therefore, the procedure does not require the operator to use a stylet, and the maneuvering of the endotracheal tube requires only that the tube to be pushed inside a stiff routing channel.

Although this solution prevents possible risks arising from the use of stylets, and therefore represents a more comfortable procedure of orotracheal intubation, according to an indirect laryngoscopy technique, it is still characterized by significant drawbacks.

First of all, the operator is forced to express strong actions of pulling and rotating the device, just in attempting, sometimes not effectively, to align properly the far end of the instrument to the tracheal axis.

Furthermore, the requirement of inserting the endotracheal tube in an operative channel, in order to follow a proper routing path, leads as a consequence to the definition of an increased instrument's thickness, and to an increased minimum inter-foreteeth distance, so that the same instrument can enter into the patient's mouth, and/or leads to the use of different instruments of different sizes, according to the diameter of the tube to be inserted.

### SUMMARY OF THE INVENTION

Therefore, the subject of the present invention consists of a device overcoming all the previous drawbacks, and achieving a great versatility of use, because it can be embedded into a large portion of the available systems supporting the orotracheal intubation actually on the market, like i.e.: laryngoscopes, videolaryngoscopes, tunneled videolaryngoscopes, etc.

In particular, the device of this invention achieves a guiding and routing system for an endotracheal tube by use of one or more planar elements, each having a profile that is perfectly adapted to the curvature of said blade in which it is placed, and from which it can protrude out, on command, through a groove, in order to achieve one or more respective fixed transversal wall.

Another significant feature of the present invention is the possibility of integration in a guiding system by magnetic means, according to concepts and disclosure from the European patent application EP 11425045 (De Domenico), so that the intubation procedure can be adapted to different operative circumstances, especially in the case where a direct or indirect visual perception of the situation is possible. More specifically, the above integration would achieve a synergy of the two systems, the first based on planar elements and the second based on magnetic elements, so that a smaller size of both devices is possible, according to smaller planar elements and smaller magnetic elements, without affecting the effectiveness of instruments, and improving instead the general efficiency due to the decreased length of inter-foreteeth distance.

The disclosed device permits therefore the achievement of a significant decrease of thickness, a variable routing direction at each step of the procedure, under control of a guiding mechanism, and a more comfortable intubation that does not require any use of a stylet to define shape and profile.

Therefore, it is the specific subject of the present invention a device supporting a tracheal tube, preferably applied to a blade of a laryngoscope, or an Airtraq® videolaryngoscope, or a LMA® videolaryngoscope, or other similar instruments, particularly suitable as a support to procedure of patient's tracheal intubation, and characterized in comprising:
one or more planar elements, each of them having a profile that is perfectly adapted to the curvature of said blade in which it is placed, and from which it can protrude out, on command, through a longitudinal groove, in order to achieve one or more fixed transversal walls, that are continuous or in patches; a tracheal tube that goes with its far end across said one or more transversal walls, and that can be curved and addressed towards trachea of a patient;
a set of mechanical gears that: according to a back position of a sliding button, keep said one or more planar elements placed inside the above blade; and that according to a front position of the same sliding button, push outside and distally the above said one or more planar elements, in order to achieve said one or more fixed transversal walls.

### BRIEF DESCRIPTION OF THE SEVERAL VIEWS OF THE DRAWINGS

The present invention will now be described for illustrative but not limitative purposes, according to its preferred embodiments, with particular reference to figures of the enclosed drawings, wherein:

FIG. 1 is a front perspective view of a device supporting a tracheal tube, according to the present invention, preferably embedded in a Airtraq® videolaryngoscope;

FIG. 2 is a lateral view of a device supporting a tracheal tube, embedded in a Airtraq® videolaryngoscope, like that of FIG. 1, inserted in the upper part of a patient, related to the mouth and first tract of air ways, the same patient being represented according to a lateral sectioned view;

FIGS. 3, 4 and 5 are a sequence of front perspective views of a Airtraq® videolaryngoscope, similar to that of FIG. 1, further including a set of magnetic elements, and of an endotracheal tube that is moved in contact with the same magnetic elements, that is represented at three different positions;

FIGS. 6, 8 and 10 are a sequence of front perspective views, in transparency, of a sectioned part of the blade of a Airtraq® videolaryngoscope like that of FIG. 1, where the inner components are protruded outside, and where they are represented at three different positions;

FIGS. 7, 9 and 11 are a sequence of front perspective views, in solid, of a sectioned part of the same blade of a Airtraq® videolaryngoscope like that of FIG. 1, where the inner components are protruded outside, and distally, and where they are represented at three different positions;

FIG. 12 is a front perspective view, in transparency, of an alternative embodiment of the present invention, again applied to a sectioned part of the same blade of a Airtraq® videolaryngoscope like that of FIG. 1, where components have been moved outside, and their run has been stopped by a set of inner small cylindrical and transversal elements;

FIG. 13 is a front perspective view, in solid, of the same object represented in FIG. 12;

FIG. 14 is a front perspective view, in transparency, of an alternative embodiment of the present invention, again applied to a sectioned part of the same blade of a Airtraq® videolaryngoscope like that of FIG. 1, where components have been moved outside, and they are represented by a unique spoon-shaped convex item, and their run has been stopped by a set of inner small cylindrical and transversal elements;

FIG. 15 is a front perspective view, in solid, of the same object represented in FIG. 14;

FIG. 16 is a front perspective view, in solid, of the same object represented in FIG. 14, and of a tracheal tube that goes with its far end across said spoon-shaped item, so that said tube can be curved and addressed towards the trachea of a patient;

FIG. 17 is a front perspective view of a device supporting a tracheal tube, according to the present invention, preferably embedded in a LMA® videolaryngoscope with magnetic elements;

FIG. 18 is a rear perspective view of a device supporting a tracheal tube, according to the present invention, preferably embedded in the same LMA® videolaryngoscope with magnetic elements;

FIG. 19 is a lateral view of a device supporting a tracheal tube, applied to the same LMA® videolaryngoscope with magnetic elements, like that of FIGS. 17 and 18, inserted in the upper part of a patient, related to the mouth and first tract of air ways, the same patient being represented according to a lateral sectioned view;

FIG. 20 is a front perspective view of a device supporting a tracheal tube, according to an alternative embodiment, comprising two respective service channels: one on the right and one on the left;

FIG. 21 is a front view of a device supporting a tracheal tube, like that of FIG. 20, comprising two respective service channels: one on the right and one on the left;

FIGS. 22 and 23 are respective front views of an Airtraq® videolaryngoscope, according to another embodiment, including a set of magnetic elements and a device supporting a tracheal tube in two different positions: one housed inside and one extended outside.

### DETAILED DESCRIPTION

It is here underlined that only a few of the many conceivable embodiments of the present invention are described, which are just some specific non-limiting examples, having the possibility to describe many other embodiments based on the disclosed technical solutions of the present invention.

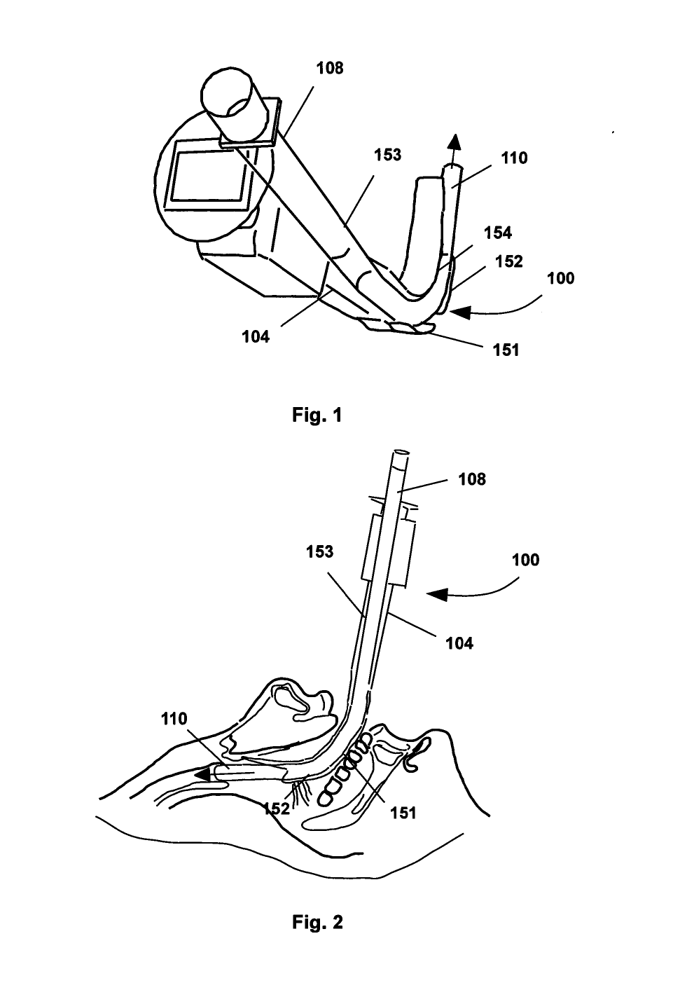

In FIG. 1 is illustrated a Airtraq® videolaryngoscope 100, particularly suitable as a support to the procedure of tracheal intubation of a patient. The blade 104, of the same videolaryngoscope 100, in the prior art has a longitudinal profile with some fixed protruding walls 151, 152, 154, that achieve a guiding and routing tunnel, along which an endotracheal tube 108 is inserted up to its final position, with the distal end in the trachea, as illustrated in FIG. 2.

Instead, device 100 of the present invention is characterized by having the protruding walls partially or completely removed. More exactly, the upper wall 154 has been partially removed, so that it can still push the upper tissues and tongue of patient, and the lower wall has been completely removed. As a consequence of having this new geometrical shape, the device 100 results in being smaller, and its insertion in the first tract of a patient's air ways results in being more comfortable to medical doctors, and at the same time it decreases the risk of wrong and/or traumatic insertions.

Indeed, when device 100 is placed at its final position, the function guiding a tracheal tube 108 is achieved again, by a mobile protruding wall 151, 152, that is pushed outside and forward, through a longitudinal groove of blade 104. The wall is composed by one or more planar elements 151, 152, each of them having a profile that is perfectly adapted to the curvature of said blade in which it is placed. For this reason, elements 151, 152, are preferably made using a material, or a metallic alloy, that is highly elastic and strong, like for example a flexible steel having a non-traumatic edge.

The mechanism providing a push outside, on command, of elements 151, 152, through a longitudinal groove of blade 104, includes a sliding button 153, preferably placed in comfortable position to doctors, that is in the upper part of device 100. In such a way, once the procedure of insertion of the tracheal tube 108 is started, following a lateral sliding path along the device 100, it is possible to activate said mechanism that pushes outside, simultaneously or in sequence, and moving forward, one or more elements 151, 152, in such a way as to enable one to “play off the cushion”, because the far end 110 can be curved upwards and can be addressed to the trachea of a patient. In particular, the mechanism of pushing outside and forward, on command, includes a set of mechanical gears that: according to a back position of a sliding button 153, keep said one or more planar elements 151, 152, placed inside the above blade 104; and that according to a front position of the same sliding button 153, push outside and forward the above said one or more planar elements 151, 152, outside and distally, in order to achieve the fixed transversal wall.

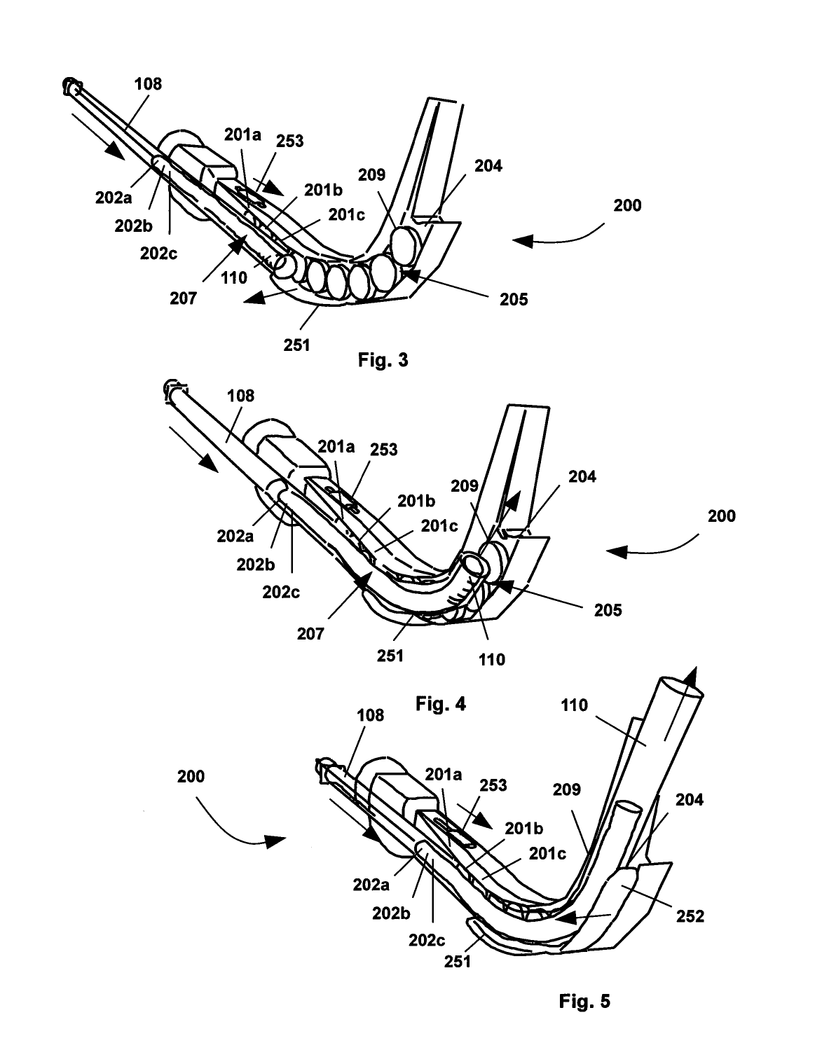

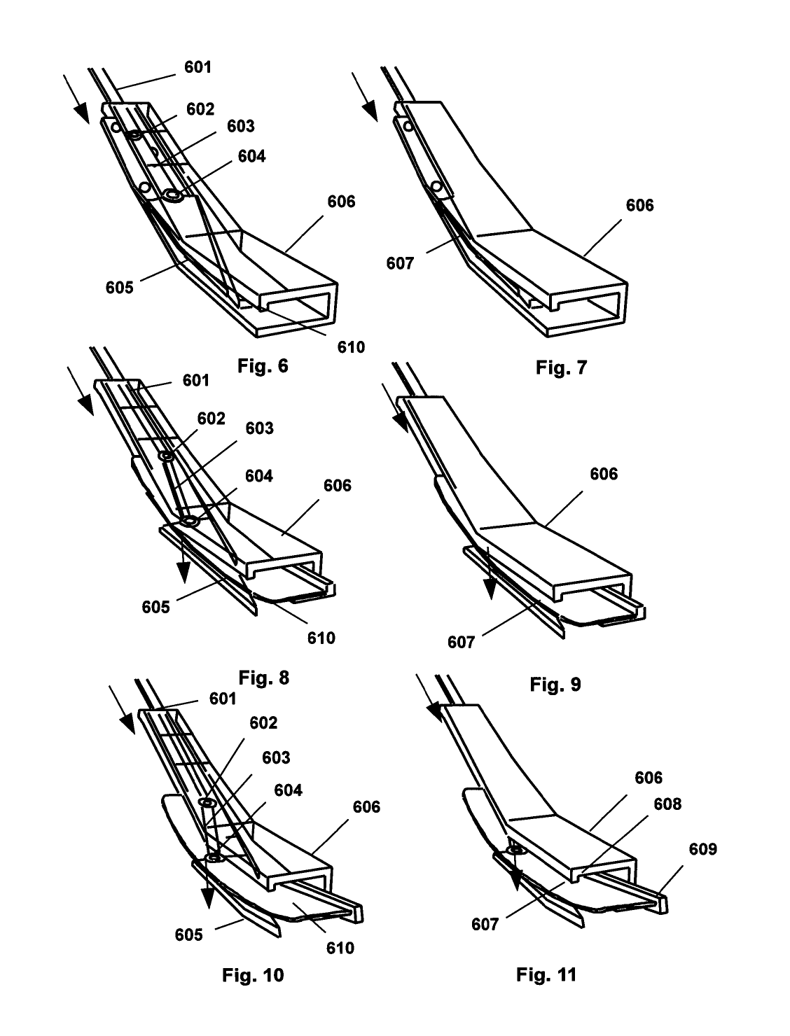

FIGS. 3, 4 and 5 are a sequence of front perspective views of a Airtraq® videolaryngoscope 200, similar to that of FIG. 1, further including a set of magnetic elements, according to concepts and disclosures from the European patent application EP 11425045 (De Domenico), filed Feb. 23, 2011.

In device 200 the guiding function is achieved through a magnetic platform 205, that is placed into the blade, and that interacts with a magnetic train 207, that has been previously and steadily inserted within an endotracheal tube 108.

The magnetic platform 205 comprises a first set of magnetic elements 201a, 201b, . . . , etc., that are all connected to each other, by a first flexible supporting structure; the magnetic train 207 comprises instead a second set of magnetic elements 202a, 202b, . . . , etc., that are all connected to each other, by a second flexible supporting structure. The elements 202a, 202b, . . . , etc., of the magnetic train 207 have an opposite polarity with respect to elements 201a, 201b, . . . , etc., of said magnetic platform 205, so that a magnetic attractive force is generated by a constant interaction, keeping said endotracheal tube 108 strictly in contact with said magnetic platform 205.

Therefore, FIGS. 3, 4 and 5 show a sequence where an endotracheal tube 108 is moved in contact with the magnetic platform 205, and it is represented at three different positions.

An operator can move the endotracheal tube 108, forward or backward, along the longitudinal profile of said operative channel 204, in order to achieve a guiding and routing system of the same endotracheal tube 108, because the tube follows a respective curved trajectory and it extends itself with its far end 110 aligned to a direction that follows the tangent to said operative channel's 204 profile, at the last point of magnetic contact 209.

Therefore, the guiding system of the present invention is integrated in said magnetic guiding system, already known from the prior art, and it includes a mobile protruding wall 251, 252, that is pushed outside, through a longitudinal groove, from the blade of the same device 200.

In FIG. 3 is shown that a forward motion of sliding button 253 activates a mechanism, which is smooth or by click, pushing outside first the protruding element 251, like in FIG. 4, and then the protruding element 252, like in FIG. 5. In such a way, elements 251, 252, enable the tracheal tube 108 to “play off the cushion”, because the far end 110 can be curved upwards and can be addressed to the trachea of a patient.

Said mechanism pushing outside each protruding element is explained now more in detail, with reference to FIGS. 3, 4 and 5.

The object shown in the same FIGS. 3, 4 and 5 is a sectioned part of the blade 606 of a Airtraq® videolaryngoscope, or other similar instruments, having a squared cross-section, with a hollow inside, where a triangle-based support 605 is housed. This support 605 can move laterally outside of blade 606, following a motion transmitted by a rod 601 connected to it. In the upper part, the same rod 601 is connected to a sliding button (not illustrated).

A motion forward of said button causes the motion forward of rod 601, following the direction of the arrow and, through an articulated tract 603 and pivots 602 and 604, the same motion is transmitted to support 605, sliding outside of blade 606, along a lateral oblique wall, in touch to another triangle-based and fixed element 610. A protruding element 607 is connected on the top of said support 605, and represents a wall that, touched by the far end of a tracheal tube, enables it to “play off the cushion”, changing and curving the insertion trajectory of the same tracheal tube.

The protruding element 607 has a transversal edge 609 on the inner side, that is intercepted by a respective edge 608 on the profile of blade 606, so that an end-run function is embedded in the motion of said element 607.

FIG. 16 shows more in detail how a far end 110 of a tracheal tube 108, that touches element 709, causes its trajectory to be curved upwards.

The above described mechanism allows, therefore, a more comfortable and effective maneuvering for doctors, in order to achieve a proper tracheal intubation of patients.

The mechanism pushing outside the protruding element 607 is perfectly reversible, and that means that a motion backward of said sliding button causes a motion backward of said rod 601 following a direction opposite to the arrow and, through the same articulated tract 603 and pivots 602 and 604, the motion is transmitted to support 605, sliding inside of blade 606, along a lateral oblique wall, in touch with the triangle-based and fixed element 610.

FIGS. 7, 9 and 11 show the same elements and mechanisms, in solid, so that a condition of real use of the device is represented.

The described mechanism can include, without loss of generality, more protruding elements, for example two or three, that are pushed outside, on command, from the blade of the device, sequentially or simultaneously, and that are installed at different distances, along the longitudinal axis of the blade. Furthermore, said protruding elements can lie on different levels, one with respect to the others, and can possibly be placed with a certain degree of overlap. Again, the same protruding elements can be installed on different supporting structures, for example: one set on the right side and another set on the left side with respect to a channel where a tracheal tube is inserted.

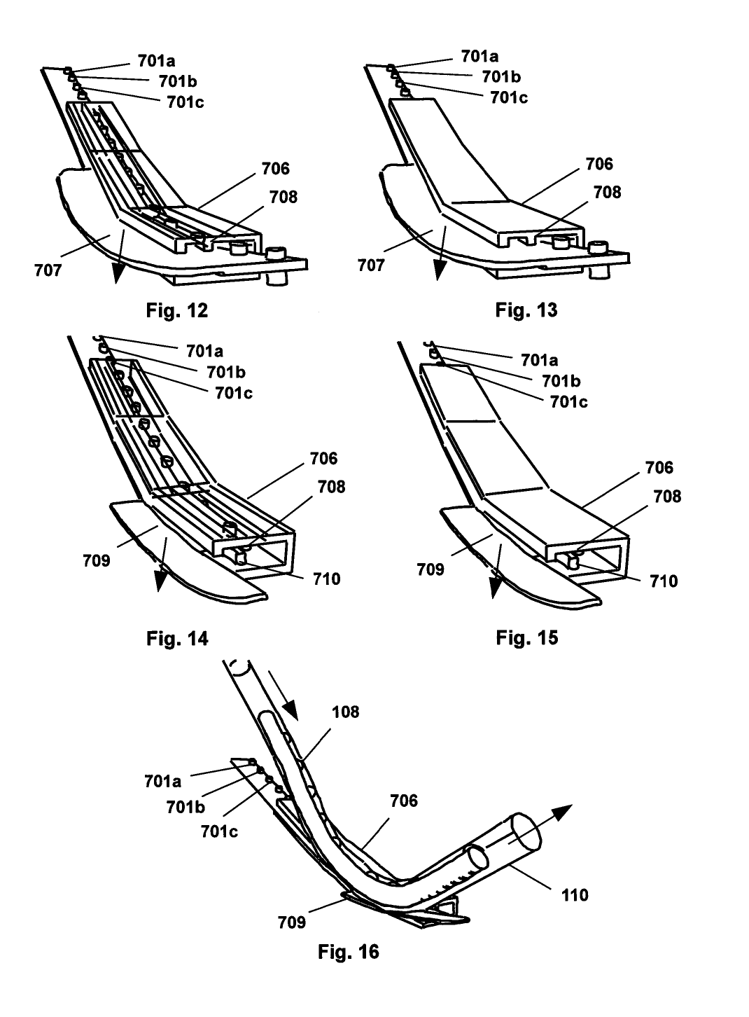

FIG. 12 shows, in transparency, an alternative embodiment of the present invention, applied to a blade 706, where the protruding element 707 is composed, indeed, by a unique flexible plate, having a set of small cylinders 701a, 701b, 701c, etc., installed on it following a longitudinal line, so that when they intercept the edge 708 they embed the same function of end-run, as previously described.

FIG. 14 shows, in transparency, another alternative embodiment of the present invention, similar to the previous one, where components 709 are represented by a unique spoon-shaped convex item. Again, a set of small cylinders 701a, 701b, 701c, etc., are installed on it following a longitudinal line, and when they intercept the edges 708 and 710 they embed the same function of end-run, as previously described.

FIGS. 13 and 15 show the same elements and mechanisms, in solid, so that a condition of real use of the device is represented.

The mechanism controlling the above action of pushing outside the protruding elements, or pulling them inside the blade, and/or their simultaneous motion forward or backward along the longitudinal axis of the same blade, can further include additional gears, defining and enabling a “click” mechanism. These additional gears can include springs that are charged, with stopping clips that, when removed by a command button, cause the protruding wall to be immediately “clicked” outside, or “clicked” inside.

Further embodiments of the invention can include more mechanisms, one for each protruding element, that can be activated simultaneously, or sequentially, and the related technical changes are immediate to those skilled in the art, in order to obtain medical devices that are comfortable and easy to be used.

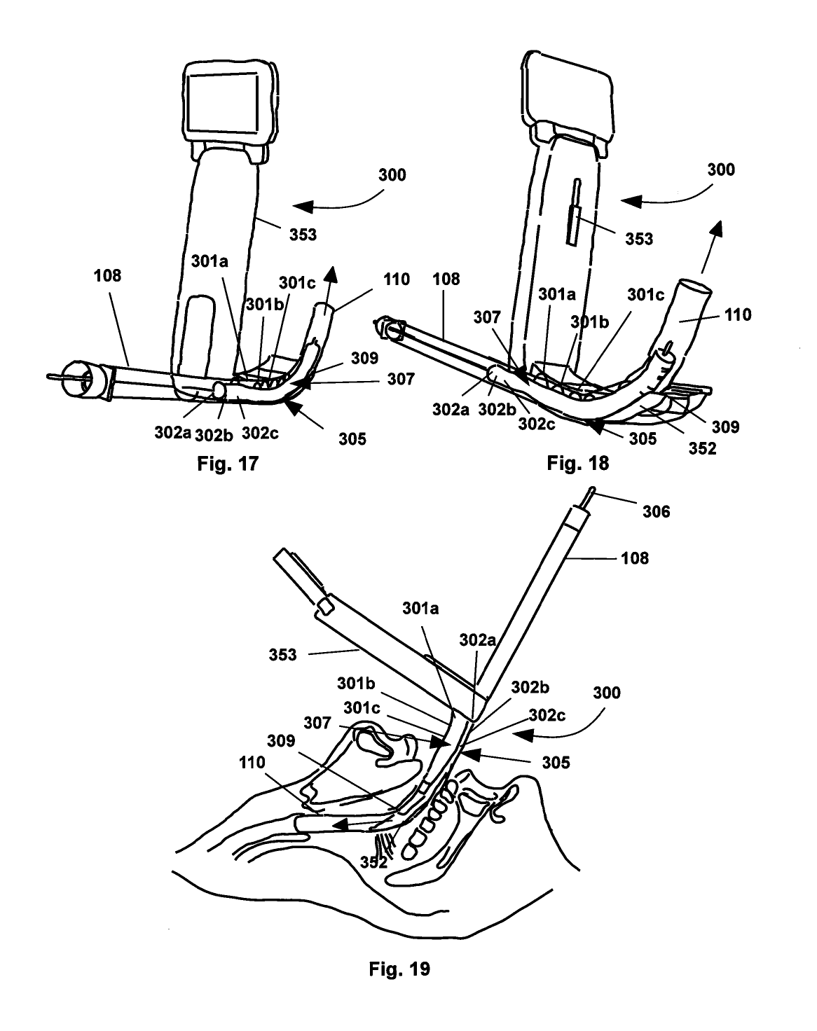

FIGS. 17 and 18, show according to two different angles, another alternative embodiment of the present invention, applied to the blade of a LMA® videolaryngoscope 300 with magnetic elements, according to concepts and disclosures from the European patent application EP 11425045 (De Domenico), filed Feb. 23, 2011.

In device 300 the guiding function is achieved through a magnetic platform 305, that is placed into the blade, and that interacts with a magnetic train 307, that has been previously and steadily inserted within an endotracheal tube 108.

The magnetic platform 305 comprises a first set of magnetic elements 301a, 301b, . . . , etc., that are all connected to each other, by a first flexible supporting structure; the magnetic train 307 comprises instead a second set of magnetic elements 302a, 302b, . . . , etc., that are all connected to each other, by a second flexible supporting structure. The elements 302a, 302b, . . . , etc., of the magnetic train 307 have an opposite polarity with respect to elements 301a, 301b, . . . , etc., of said magnetic platform 305, so that a magnetic attractive force is generated by a constant interaction, keeping said endotracheal tube 108 strictly in contact with said magnetic platform 305.

An operator can move the endotracheal tube 108, forward or backward, along the longitudinal profile of the laryngoscope's blade, in order to achieve a guiding and routing system of the same endotracheal tube 108, because the tube follows a respective curved trajectory and it extends itself with its far end 110 aligned to a direction that follows the tangent to said laryngoscope's profile, at the last point of magnetic contact 309.

Therefore, the guiding system of the present invention is integrated in said magnetic guiding system, already known from the prior art, and it includes a mobile protruding wall 352, that is pushed outside and smoothly forward, through a longitudinal groove, from the blade of the same device 300.

A forward motion of sliding button 353 activates a mechanism, which is smooth or by click, pushing outside and forward the protruding element 353. In such a way, elements 352 enable the tracheal tube 108 to “play off the cushion”, because the far end 110 can be curved upwards, smoothly and in an adjustable way, and can be addressed to the trachea of a patient, as illustrated in FIG. 19.

The supporting device, subject of the present invention, can be easily integrated with optical means, typical of a Airtraq® videolaryngoscope, or a LMA® videolaryngoscope, or other similar instruments.

For example, the housing containing the mechanisms that push said protruding element outside, can be perfectly parallel to a visual optical channel, through which the visual signals of the detected image are transmitted, from the far end of the laryngoscope.

Furthermore, some embodiments of the invention disclose that said operative channel can be placed at the right side, from the point of view of an operator, or at the left side. Then, another embodiment of invention discloses that said operative channel, along which said endotracheal tube 108 is inserted, can be central and parallel with respect to the two service channels, one of them being for example an optical visual channel, and the other the one where said protruding element is contained.

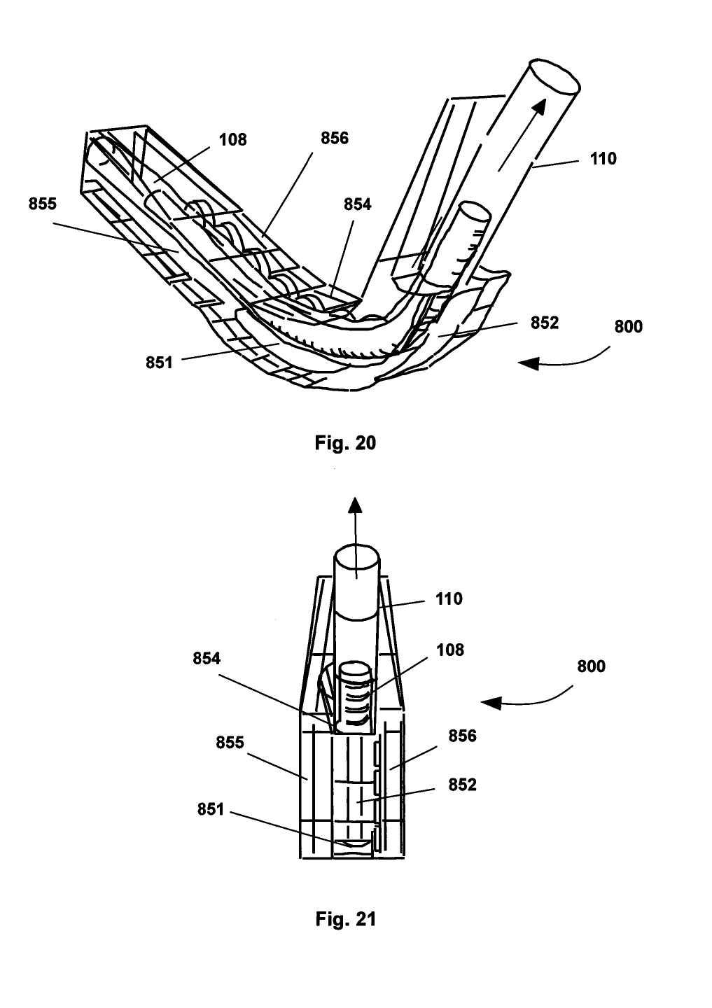

Furthermore, according to another embodiment 800 of the present invention, shown in FIGS. 20 and 21, the operative channel 854, along which the endotracheal tube 108 is inserted, can be placed centrally with respect to two lateral channels 855, 856, each of them being divided into at least two semi-channels. These lateral semi-channels are particularly suitable as respective housings of two sets of protruding elements 851, 852 (one on the right and one on the left), with the other two semi-channels being used: one to contain an optical visual system, the other one to contain any other device, for example, a magnetic platform, or a more simple rod that, being placed under one of the semi-channels containing the protruding elements 851, 852 (and ending with the distal part close to the far end of blade) is able to curve/sustain the far end of blade, achieving the same function as a McCoy laryngoscope.

In such a way, the patient's anatomical parts result in being better protected, with respect to situations where the endotracheal tube 108 could touch the tongue and tissues, with risk of abrasions and inner scratching for the patient.

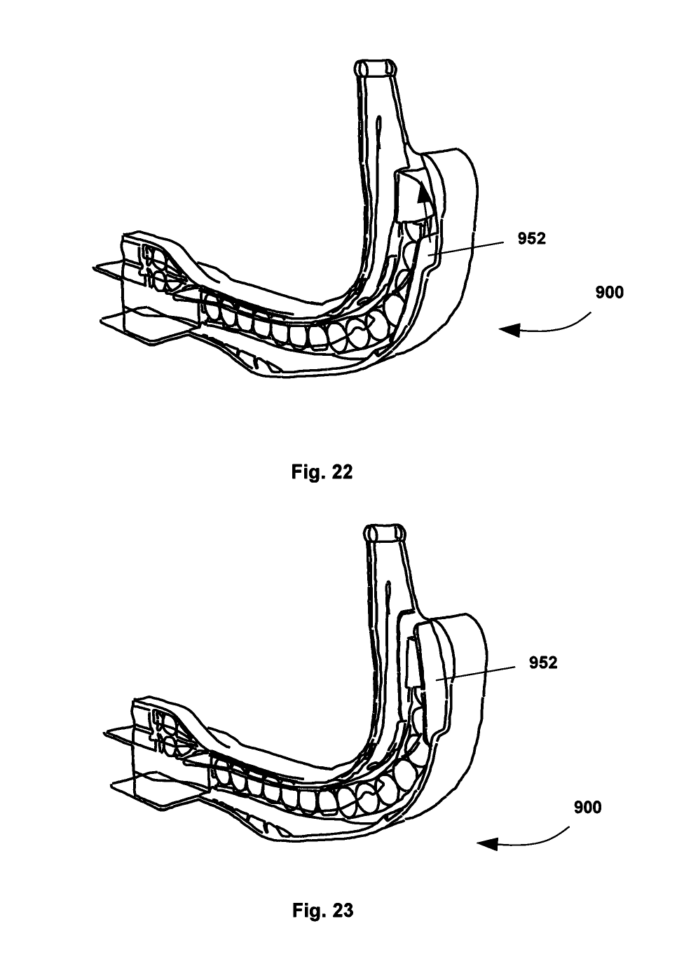

FIGS. 22 and 23 show respective front views of an Airtraq® videolaryngoscope 900, according to another embodiment, including a set of magnetic elements and a device 952 supporting a tracheal tube in two different positions: one housed inside and one extended outside.

Therefore, the above examples show that the present invention achieves all the proposed objectives. In particular, it permits one to obtain a device that permits the overcoming of all of the drawbacks of the prior art, achieving a great versatility of use, because it can be embedded into a large portion of the available systems supporting the orotracheal intubation, actually on the market, like i.e.: laryngoscopes, videolaryngoscopes, tunneled videolaryngoscopes, etc.

In particular, the device of this invention achieves a guiding and routing system for an endotracheal tube by use of one or more planar elements, each having a profile that is perfectly adapted to the curvature of said blade in which it is placed, and from which it can protrude out, on command, through a groove, in order to achieve one or more respective fixed transversal wall.

Another significant feature of the present invention is the possibility of integration in a guiding system by magnetic means, according to concepts and disclosure from the European patent application EP 11425045 (De Domenico), so that the intubation procedure can be adapted to different operative circumstances, especially in the case where a direct or indirect visual perception of the situation is possible. More specifically, the above integration would achieve a synergy of the two systems, the first based on planar elements and the second based on magnetic elements, so that a smaller size of both devices is possible, according to smaller planar elements and smaller magnetic elements, without affecting the effectiveness of instruments, and improving instead the general efficiency due to the decreased length of inter-foreteeth distance.

Then, the disclosed device permits therefore the achievement of a significant decrease of thickness, a variable routing direction at each step of the procedure, under control of a guiding mechanism, and a more comfortable intubation that does not require any use of a stylet to define shape and profile.

The present invention has been described for illustrative but not limitative purposes, according to its preferred embodiments, but it is clear that modifications and/or changes can be introduced by those skilled in the art without departing from the relevant scope, as defined in the enclosed claims.

1. A device for supporting a tracheal tube, preferably applied to a curved blade of a laryngoscope, videolaryngoscope, or other similar instrument, and particularly suitable as a support for the procedure of tracheal intubation of a patient, the device comprising:
one or more planar elements placed in said blade, each of the one or more planar elements having a profile that is adapted to the curvature of said blade, and being further adapted for protruding out, on command, through a longitudinal groove in said blade, in order to form one or more fixed transversal walls, said walls being continuous or in patches;
a mechanism that includes a set of mechanical gears that, according to a back position of a sliding button, keep said one or more planar elements placed inside said; and that, according to a front position of the sliding button, push said one or more planar elements outside and distally in order to force said one or more planar elements to protrude through said longitudinal groove and form said one or more fixed transversal walls;
so that as a distal end of the tracheal tube moves across said one or more transversal walls, said tube is curved and addressed towards a trachea of a patient.

2. A device for supporting a tracheal tube, according to claim 1 wherein,
the mechanism that allows the one or more planar elements to be pushed out, on command, through the longitudinal groove of the blade, further includes a triangle-based support; the support being adapted for moving laterally outside of said blade, following a motion transmitted by a rod connected to it; the rod having an upper part is connected to the sliding button;
such that a motion forward of said button causes a motion forward of said rod through an articulated tract and pivots, the same motion thereby being transmitted to the support, causing the support to slide outside of the blade, along a lateral oblique wall, and along a triangle-based and fixed element; and
a protruding element connected on a top of said support, the protruding element representing a wall that, when touched by the far end of the tracheal tube, enables the tube to “play off the cushion”, thereby changing and curving the insertion trajectory of the tube.

3. A device for supporting a tracheal tube, according to claim 2, wherein:
the protruding element has a transversal edge on an inner side, said inner side being intercepted by a respective edge on a profile of the blade during the motion of the protruding element, so that an end-run function is embedded in the motion of said protruding element.

4. A device for supporting a tracheal tube, according to claim 2, wherein:
said mechanism for pushing outside the protruding element is reversible, such that a motion backward of said sliding button causes a motion backward of said rod through the articulated tract and pivots, the same backward motion thereby being transmitted to the support, causing the support to slide inside of the blade, along the lateral oblique wall, and along the triangle-based and-fixed element.

5. A device for supporting a tracheal tube, according to claim 1, wherein:
said planar elements, comprise a material, or a metallic alloy, that is highly elastic and strong.

6. A device for supporting a tracheal tube, according to claim 1, wherein:
said device is integrated with a magnetic guiding system, a guiding function of said system being achieved through a magnetic platform that is placed into the blade and that interacts with a magnetic train that has been previously and steadily inserted within the tube.

7. A device for supporting a tracheal tube, according to claim 6, wherein:
The magnetic platform comprises a first set of magnetic elements connected to each other by a first flexible supporting structure; the magnetic train comprises instead a second set of magnetic elements connected to each other by a second flexible supporting structure;
the first set of magnetic elements having an opposite polarity with respect to the second set of magnetic elements, so that a magnetic attractive force is generated by an interaction between the first and second magnetic elements, thereby keeping said tracheal tube strictly in contact with said magnetic platform.
so that an operator can move the tracheal tube, forward or backward, along a longitudinal profile of an operative channel, in order to achieve the guiding function of the magnetic guiding system of the tube, because the tube follows a respective curved trajectory and it extends itself with its distal end aligned to with a direction that follows the tangent to said operative channel's profile, a last point of magnetic contact.

8. A device for supporting a tracheal tube, according to claim 1, further comprising:
a protruding element comprising a flexible plate extending longitudinally inside the blade, the protruding element representing a wall that, when touched by the far end of the tracheal tube, enables the tube to “play off the cushion”, thereby changing and curving the insertion trajectory of the tube; and
the plate having a set of small cylinders installed on it following a longitudinal line, so that during a forward motion of the flexible plate, the set of cylinders are intercepted by a respective edge on a profile of the blade, whereby an end-run function is embedded in the motion of said protruding element.

9. A device for supporting a tracheal tube, according to claim 1, further comprising:
a protruding element comprising a unique spoon-shaped convex item extending longitudinally inside the blade, the protruding element representing a wall that, when touched by the far end of the tracheal tube, enables the tube to “play off the cushion”, thereby changing and curving the insertion trajectory of the tube; and
the protruding element having a set of small cylinders installed on it following a longitudinal line, so that during a forward motion of the convex item, the set of cylinders are intercepted by a respective edge on a profile of the blade, whereby an end-run function is embedded in the motion of said protruding element.

10. A device for supporting a tracheal tube, according to claim 1, wherein:
the mechanism that allows the one or more planar elements to be pushed out, on command, through the longitudinal groove of the blade includes additional gears, the additional gears defining and enabling a “click” mechanism and including springs that are charged, with stopping clips that, when removed by a command button, cause the protruding portion of the planar elements to be immediately “clicked” outside, or “clicked” inside the blade.

11. A device for supporting a tracheal tube, according to claim 10, wherein:
said device includes a plurality of additional mechanisms, such that there is one mechanism for each of said planar elements, that can be activated simultaneously, or sequentially, in order to obtain a medical device that is comfortable and easy to be used.

12. A device for supporting a tracheal tube, according to claim 1, wherein:
the mechanisms that allows the one or more planar elements to be pushed out, on command, through the longitudinal groove of the blade, is positioned within housing, said housing being parallel to a visual optical channel through which visual signals of a detected image are transmitted, from the far end of a videolaryngoscope.

13. A device for supporting a tracheal tube, according to claim 12, wherein:
said housing is placed at one of the right side, from the point of view of an operator, or the left side of the blade.

14. A device for supporting a tracheal tube, according to claim 1, further comprising:
an operative channel along which said tube is adapted for being inserted, said operative channel being located between and parallel with respect to two service channels, one of two service channels being an optical visual channel, and the other one of the two service channels containing the one or more planar elements, so that the patient's anatomical parts are better protected, with respect to situations where the tracheal tube could touch tongue and tissues, from a risk of abrasions and inner scratching.

15. A device for supporting a tracheal tube, according to claim 1 further comprising:
an operative channel along which the tracheal tube is adapted for being inserted, said operative channel being placed centrally with respect to two lateral channels, each of the two lateral channels being divided into two semi-channels; one of the two semi-channels located to the left of the operative channel housing a first set of protruding portions of the one or more planar elements; one of the two semi-channels located to the right of the operative channel housing a second set of protruding portions of the one or more planar elements; a third of the semi-channels containing an optical visual system; and a fourth of the semi-channels containing a rod that, being placed under one of the semi-channels containing the protruding portions of the planar elements and having a distal part placed close to a distal end of the blade, is able to curve and sustain the distal end of the blade.
